(19) United States
(12) Patent Application Publication
(10) Pub. No.: US 2011/0281810 A1
(43) Pub. Date: Nov. 17, 2011

(54) Composition

(75) Inventors: Kirsten Petersen, Kiel (DE); Bernd W. Müller, Flintbek (DE); Thomas Rosen, Neuss (DE)

(73) Assignee: Cognis IP Management GmbH, Dusseldorf (DE)

(21) Appl. No.: 13/086,210

(22) Filed: Apr. 13, 2011

(30) Foreign Application Priority Data
Apr. 14, 2010 (EP) 10003932.0

(51) Int. Cl. A61K 47/10; A61K 47/14; A61K 31/5513; A61P 15/12; A61K 31/496; A61P 25/20; A61P 31/00; A61P 19/10; C07C 43/13; A61K 31/7048

(52) U.S. Cl. 514/29; 568/625; 514/786; 514/772; 514/221; 514/254.07

(57) ABSTRACT

The present invention relates to the use of a solubilizer for enhancing the solubility of an active pharmaceutical ingredient in water. The present invention further relates to a composition comprising a solubilizer and at least one active pharmaceutical ingredient. In both cases the solubilizer is an alkoxylated monoalcohol or a mixture of alkoxylated monoalcohols, where the alkoxylated monoalcohol or, if a mixture of alkoxylated monoalcohols is present, at least one alkoxylated monoalcohol in the mixture comprises at least one oxypropylene unit.

### CROSS-REFERENCE TO RELATED APPLICATIONS

This application claims the benefit of priority under 35 U.S.C. §119(a) to European Patent Application No. 10003932.0, filed Apr. 14, 2010, which is hereby incorporated by reference in its entirety.

The present invention relates to the use of a solubilizer for enhancing the solubility of an active pharmaceutical ingredient in water. The present invention further relates to a composition comprising a solubilizer and at least one active pharmaceutical ingredient. In both cases the solubilizer is an alkoxylated monoalcohol or a mixture of alkoxylated monoalcohols, where the alkoxylated monoalcohol or, if a mixture of alkoxylated monoalcohols is present, at least one alkoxylated monoalcohol in the mixture comprises at least one oxypropylene unit.

A solubilizer is a compound or mixture of compounds that serves to enhance the solubility of a compound whose solubility in water is limited or even very sparing.

Alkoxylated monoalcohols, especially fatty alcohol polyglycol ethers, are known surfactants. They are used as solubilizers, among other applications, in the cosmetic sector, among other sectors. Fatty alcohol polyglycol ethers are described in Chemielexikon Römpp Online, Version 3.6 (Georg Thieme Verlag; http://www.roempp.com). Fatty alcohols are linear, saturated or unsaturated primary alcohols having 6 to 22 C atoms.

The use of ethoxylated monoalcohols not containing an oxypropylene unit as solubilizers for active pharmaceutical ingredients is known from, for example, European Journal of Pharmaceutics and Biopharmaceutics, Volume 51, pages 221-226 (2001) and International Journal of Pharmaceutics, Volume 92, pages 191-196 (1993) and J. Chem. Eng. Data, Volume 53, pages 1271-1277 (2008) and Journal of Pharmaceutical Science and Technology, Volume 52, pages 33-36 (1998).

US 2009/311195 discloses, more particularly in claim 1, various compositions comprising a surfactant and an active component. A specific composition comprising an alkoxylated monoalcohol which contains at least one oxypropylene unit and an active pharmaceutical ingredient is not disclosed.

One possibility for the naming of fatty alcohol polyglycol ethers will be illustrated using the following example. Polyoxypropylene-1-polyoxyethylene-9 lauryl ether is obtainable by ethoxylating and propoxylating lauryl alcohol (1-dodecanol) and has on average (number average) one oxypropylene unit and nine oxyethylene units. The alkoxylation may be accomplished by means of a mixture of ethylene oxide and propylene oxide, or may take place in two stages, by alkoxylation first with ethylene oxide and then with propylene oxide, or vice versa, or may take place as a multistage operation. In other words, the name polyoxypropylene-1-polyoxyethylene-9 lauryl ether states only that per molecule there are on average one oxypropylene unit and nine oxyethylene units, without stating the order in which these units are present in the molecule.

Increasing numbers of active ingredients on the pharmaceutical market exhibit poor solubility in water and often, concomitantly, poor bioavailability. In order to be able to administer these active ingredients in an appropriate form, it is necessary to bring them more effectively into solution, by a variety of methods. Particularly in the area of liquid presentation forms, however, additional requirements, such as minimal administration volumes for sufficient amounts of drug, and low toxicity, are often difficult to meet. For this reason, solubilization represents an important area in the development of new drug forms.

There are a host of chemical and technological measures in existence for altering the solubility of active ingredients. One of these methods is the solubilization of sparingly soluble active ingredients, where surface-active substances are used to improve solubility. These surface-active substances are composed of hydrophilic and lipophilic moieties which are usually present in separate areas of the molecule. For this reason they are also referred to as amphiphilic substances. The surfactant molecules congregate to form what are called micelles, which have a lipophilic region in the interior but are outwardly hydrophilic. Lipophilic compounds can be held in this lipophilic interior, and so clear solutions are formed.

A number of companies are active in the field of the solubilization of active ingredients. They include manufacturers of pharmaceutical excipients, such as BASF, Gattefossé, Croda or Evonik. Within the pharmaceutical industry, generally speaking, there is only a limited selection used of solubilizers that have already been monographed in the various pharmacopoeias. In contrast, there are a series of solubilizers and synergistic mixtures of solubilizers for cosmetic application.

As described, sparingly soluble active ingredients pose a major problem in pharmaceutical development, since the bioavailability of these substances is generally poor and hence their activity is reduced, in some cases extremely. There is a limited selection of solubility-enhancing substances which are able to enhance the bioavailability of such sparingly soluble compounds. In many cases, however, this selection is not sufficient to enhance the solubility of new active ingredients in such a way that they can be employed therapeutically. A further problem, moreover, are in some cases toxic properties of solubilizers.

The object on which the present invention was based was that of providing further solubilizers for active pharmaceutical ingredients.

This object has been achieved through the provision of the solubilizers defined in the claims for the solubilization of active pharmaceutical ingredients.

The present invention first provides, therefore, for the use of a solubilizer for enhancing the solubility of an active pharmaceutical ingredient in water, as defined in the claims.

The present invention further provides a composition as defined in the claims.

The dependent claims show particular embodiments of the present invention.

The alkoxylated monoalcohols for use in accordance with the invention may have only one fatty alcohol radical, e.g., the lauryl residue, or a mixture of different fatty alcohol residues. For the alkoxylation it is possible in particular to use fatty alcohols based on natural raw materials. For example, what is called cocoyl alcohol can be used. Cocoyl alcohol is a fatty alcohol mixture obtainable from coconut oil and composed in the greatest part of lauryl alcohol.

In accordance with the invention, alkoxylation with a mixture of two or more alkoxides may mean either that an alcohol or an alcohol mixture is reacted with a mixture of two or more alkoxides or that an alcohol or an alcohol mixture is first reacted with an alkoxide and the reaction product obtainable in this reaction is then reacted with a second and subsequently, if desired, with a third alkoxide.

It has been found that the alkoxylated monoalcohols of the invention which comprise at least one oxypropylene unit are better solubilizers than the alkoxylated monoalcohols known from the prior art, which are, for example, ethoxylated lauryl alcohols containing no oxypropylene unit.

### EXAMPLES

% figures denote % by weight in each case unless otherwise specified.

A range of amphiphilic substances employed to date only in cosmetic formulations were investigated for use as solubilizers for active pharmaceutical ingredients. These solubilizers have not to date been used for enhancing the solubility of active pharmaceutical ingredients.

The products in question are as follows:
Eumulgin® ES: PPG-5-Laureth-5 (INCI name)
Eumulgin® L: PPG-1-PEG-9 Lauryl Glycol Ether (INCI name)
Eumulgin® HPS: mixture of 25-50% Coceth-7, 25-50% Eumulgin® L, 10-20% Eumulgin®HRE PH, and 5-10% water

The structure of the stated products is clear through the following naming:
Eumulgin® ES: polyoxypropylene-5 polyoxyethylene-5 lauryl ether
Eumulgin® L: polyoxypropylene-1 polyoxyethylene-9 lauryl ether
Eumulgin® HPS: 25-50% polyoxyethylene-7 cocoyl ether, 25-50% polyoxypropylene-1 polyoxyethylene-9 lauryl ether, 10-20% polyoxyethyleneglycerol trihydroxystearate 40, and 10-20% water

These products are all available from Cognis GmbH, Monheim, Germany.

The results found in these investigations were as follows:
All of the stated solubilizers can be used technically in pharmaceutical formulations. They have physical properties (viscosity, CMC, and micelle size) which suggest no problems with their use.
Eumulgin® HPS: This synergistic mixture of different products produced a significant dissolution improvement for various active ingredients. The enhanced solubilities are in many cases above that of other solubilizers established on the market.
Eumulgin® HPS results in significantly higher solubility improvements for diazepam, which is above that of other standard market products such as Solutol® HS15 (BASF SE, Ludwigshafen, Germany), Eumulgin® RO 35 PH or Polysorbat® 80.
Eumulgin® L is a significantly more effective solubilizer for estradiol than other standard market products such as Solutol® HS15 (BASF), Eumulgin® RO 35 PH or Polysorbat® 80.
Eumulgin® ES is a significantly more effective solubilizer for various cinnamic acid derivatives such as butyl cinnamate and phenethyl cinnamate than other standard market products such as Solutol® HS15 (BASF), Eumulgin® RO 35 PH or Polysorbat® 80.
For pharmaceutical applications, moreover, it is relevant that all of the solubilizers enhanced the solubility of the model compounds in water. The concentrations achieved are beyond the concentration present in market products. This is relevant, since it allows cosolvents such as ethanol to be avoided, which can have an irritant effect and may have other toxic side-effects, particularly in application in infants.
All the solubilizers are already established in cosmetic applications and characterized by high compatibility and low toxicity.

The stated products and synergistic mixtures were investigated for their usefulness in pharmaceutical formulations and in particular for solubility improvement for sparingly soluble model substances. Active ingredients used in these investigations were the following compounds: diazepam, erythromycin, estradiol, itraconazole. The selected model drugs exhibit poor solubility in water. In order to cover as broad a spectrum of drugs as possible, they come from chemically different groups and are active ingredients that are in common use.

The effect of the stated new solubilizers on the boost to solubility of these active ingredients was investigated and was compared with solubilizers already established for pharmaceutical applications. For this purpose, solution isotherms were recorded, and can be used to ascertain the extent of dissolution mediated by the various solubilizers. The optimum is a sharp increase in drug concentration through the use of small amounts of solubilizer, since increasing concentration may also be accompanied by an increase in the unwanted effects.

The table below shows the enhancement in solubility of various sparingly soluble active ingredients through a series of solubilizers. The model substances selected in this case were diazepam, erythromycin, itraconazole, and estradiol. The solubilizers investigated were compared with solubilizers already established for pharmaceutical formulations, such as TEGO® SMO 80 V (Polysorbat 80) from Evonik, Solutol® HS 15 from BASF, Ludwigshafen, and Eumulgin® RO 35 PH and Speziol® TPGS Pharma (Tocophersolan) from Cognis, Monheim.

| | | TEGO ® | | | | | | |
|---|---|---|---|---|---|---|---|---|
| | No | SMO | Solutol ® | | | Eumulgin ® | Speziol ® | |
| | solubilizer | 80V | HS 15 | Eumulgin ® L | Eumulgin ® HPS | RO35 | TPGS | Eumulgin ® ES |
| | mg/ml | mg/ml | mg/ml | mg/ml | mg/m1 | mg/ml | mg/ml | mg/ml |
| Diazepam | 0.012 | 1.817 | 1.088 | 1.866 | 2.036 | 1.765 | 1.854 | |
| Erythromycin | 0.459 | 1.695 | | 1.066 | 1.226 | 1.662 | 1.715 | |
| Itraconazole | 0.001 | 0.015 | 0.010 | 0.007 | 0.008 | 0.011 | 0.008 | 0.009 |
| Estradiol | 0.021 | 0.523 | 0.581 | 0.939 | 0.664 | 0.465 | 0.466 | 0.693 |

Additionally investigated was the solubilization of a homologous series of cinnamic acid derivatives. Cinnamic acid derivatives are model substances for active ingredients of the kind used, for example, in sun protection creams. In order to investigate a broad selection of molecules with different sizes, the following six cinnamic acid derivatives were selected: methyl cinnamate, ethyl cinnamate, isopropyl cinnamate, isobutyl cinnamate, benzyl cinnamate, and phenethyl cinnamate. All of the cinnamic acid derivatives used are virtually insoluble in water. Through the use of solubilizers it was possible to produce a significant enhancement of their solubility in water. Proving particularly effective for large and lipophilic active ingredient molecules were Eumulgin® HPS and Eumulgin® ES.

The table below shows the solubility-boosting effect of various solubilizers for a series of cinnamic acid derivatives. Cinnamic acid derivatives find use in particular as photoprotection factors in sun protection creams. The sparing solubility of these substances in such applications is frequently a problem, which can be solved by means of solubilizers.

| MW | Methyl | Ethyl | Isopropyl | Isobutyl | Benzyl | Phenethyl |
|---|---|---|---|---|---|---|
| [mg/ml] | cinnamate | cinnamate | cinnamate | cinnamate | cinnamate | cinnamate |
| Eumulgin ® HPS | 11.4 | 23.6 | 25.7 | 29.0 | 6.4 | 2.7 |
| Eumulgin ® ES | 3.0 | | 3.4 | 1.2 | 2.9 | 3.2 |
| Eumulgin ® L | 9.8 | 14.7 | 15.2 | 41.0 | 5.8 | 2.5 |
| Eumulgin ® O10 | 9.1 | 47.0 | 32.6 | 65.9 | 8.1 | 3.7 |
| Eumulgin ® RO35 | 21.5 | 68.0 | 50.9 | 54.2 | 15.3 | 4.9 |
| Speziol ® TPGS | 7.9 | 42.3 | 21.0 | 23.1 | 6.1 | 2.6 |
| Tego ® SMO 80V | 9.6 | 96.4 | 52.5 | 110.0 | 6.8 | 2.5 |

The paragraph below summarizes the results of experimentation.

Eumulgin® HPS is the best solubilizer for diazepam. Diazepam is an active ingredient from the group of the benzodiazepines. Benzodiazepines are soporifics and sedatives. Diazepam (on account of the active metabolite desmethyldiazepam) has a long half-life and is used in particular for anxiolysis, for sedation and in attacks of cramp. In the Rote Liste there are presently 13 products containing diazepam as active ingredient. They include not only solutions (for injection, for rectal administration, for oral administration) but also solid drug forms (tablets, suppositories). The concentrations of liquid solutions are between 2 mg/ml and 10 mg/ml (RoteListe® products). In these cases, generally speaking, cosolvents such as ethanol and ethanol/propylene glycol mixtures are used. Diazepam has very sparing solubility in water, and has a theoretical solubility of 12.2 μg/ml. Through the use of Eumulgin® HPS a distinct improvement was achieved in the solubility. Thus a maximum saturation solubility was found of 2.68 mg/ml in a 15% strength solution of Eumulgin® HPS in water. This figure is significantly higher than for established market solubilizers such as TEGO® SMO 80 V (1.89 mg/ml), Solutol® HS 15 (1.64 mg/ml), Eumulgin® RO35 PH (2.14 mg/ml) and Speziol® TPGS Pharma (2.23 mg/ml) for the same solubilizer concentrations.

1. A solubilizer for enhancing the solubility of an active pharmaceutical ingredient in water, the solubilizer comprising an alkoxylated monoalcohol or a mixture of alkoxylated monoalcohols, and the alkoxylated monoalcohol or monoalcohols having 6 to 30 C atoms and being linear or branched and being saturated or unsaturated and being primary, secondary or tertiary alcohols, and the alkoxylated monoalcohol or monoalcohols being obtainable by alkoxylation with ethylene oxide, propylene oxide, butylene oxide or a mixture of two or three of said alkoxides, and the alkoxylated monoalcohol or, if a mixture of alkoxylated monoalcohols is present, at least one alkoxylated monoalcohol in the mixture comprising at least one oxypropylene unit.

2. The solubilizer as claimed in claim 1, where the monoalcohol or monoalcohols have 6 to 22 C atoms.

3. The solubilizer as claimed in claim 1, where the monoalcohol or monoalcohols are saturated or unsaturated and are linear and are primary alcohols.

4. The solubilizer as claimed in claim 1, where the alkoxylated monoalcohol or monoalcohols are obtainable by alkoxylation with ethylene oxide, propylene oxide or a mixture of both alkoxides.

5. The solubilizer as claimed in claim 1, where the alkoxylated monoalcohol or monoalcohols have a number average degree of alkoxylation of 5 to 20.

6. The solubilizer as claimed in claim 1, where the alkoxylated monoalcohol or monoalcohols are selected from the group consisting of polyoxypropylene-n lauryl ether, polyoxypropylene-m-polyoxyethylene-u lauryl ether, and mixtures of two or more of these lauryl ethers, where n=5 to 20, and where the sum of m and u is 5 to 20, and where u is 1 or greater than 1.

7. The solubilizer as claimed in claim 6, where the alkoxylated monoalcohol or monoalcohols are selected from the group consisting of polyoxypropylene-1-polyoxyethylene-9 lauryl ether, polyoxypropylene-5-polyoxyethylene-5 lauryl ether and mixtures of these two lauryl ethers.

8. The solubilizer as claimed in claim 1, where the alkoxylated monoalcohol or monoalcohols are used in a mixture which further comprises polyoxyethylene-7 lauryl ether polyoxyethyleneglycerol trihydroxystearate 40, or combinations thereof.

9. A composition comprising:
the solubilizer as defined in claim 1 and at least one active pharmaceutical ingredient,
the active pharmaceutical ingredient having a solubility in water of not more than 20 per liter at 25° C.

10. The composition as claimed in claim 9, further comprising water.

11. The composition as claimed claim 9, comprising 5% to 50% by weight of the solubilizer, 0.01% to 15% by weight of the at least one active pharmaceutical ingredient, 0% to 95% by weight of water, and 0% to 90% by weight of other pharmaceutically acceptable components.

12. The composition as claimed claim 9 for application in a method for the therapeutic, including prophylactic treatment of the human or animal body.

13. The composition as claimed in claim 9, where the active pharmaceutical ingredient is diazepam, and where the composition is intended for application as a soporific or sedative in a method for the therapeutic, including prophylactic, treatment of the human or animal body.

14. The composition as claimed in claim 9, where the active pharmaceutical ingredient is erythromycin, and where the composition is intended for application as an antibiotic in a method for the therapeutic, including prophylactic, treatment of the human or animal body.

15. The composition as claimed in claim 9, where the active pharmaceutical ingredient is itraconazole, and where the composition is intended for application in a method for the therapeutic, including prophylactic, treatment of the human or animal body against climacteric symptoms or against osteoporosis.

16. The solubilizer of claim 2, wherein the monoalcohol or monoalcohols have 8 to 18 C atoms.

17. The solubilizer as claimed in claim 5, wherein the alkoxylated monoalcohol or monoalcohols have a number average degree of alkoxylation of 7 to 10.

18. The solubilizer as claimed in claim 6, where the alkoxylated monoalcohol or monoalcohols are selected from the group consisting of polyoxypropylene-n lauryl ether, polyoxypropylene-m-polyoxyethylene-u lauryl ether, and mixtures of two or more of these lauryl ethers, where n=7 to 10, and where the sum of m and u is 7 to 10.

19. The composition of claim 9, wherein the solubility in water is not more than 10.

20. A method for enhancing the solubility of an active pharmaceutical ingredient in water, the method comprising using a solubilizer in the preparation of a composition comprising the active pharmaceutical ingredient in water, wherein the solubilizer comprises an alkoxylated monoalcohol or a mixture of alkoxylated monoalcohols, and the alkoxylated monoalcohol or monoalcohols having 6 to 30 C atoms and being linear or branched and being saturated or unsaturated and being primary, secondary or tertiary alcohols, and the alkoxylated monoalcohol or monoalcohols being obtainable by alkoxylation with ethylene oxide, propylene oxide, butylene oxide or a mixture of two or three of said alkoxides, and the alkoxylated monoalcohol or, if a mixture of alkoxylated monoalcohols is present, at least one alkoxylated monoalcohol in the mixture comprising at least one oxypropylene unit, and the active pharmaceutical ingredient has a solubility in water of not more than 20.
